(19) United States
(12) Patent Application Publication
(10) Pub. No.: US 2013/0324564 A1
(43) Pub. Date: Dec. 5, 2013

(54) POLYMORPHS OF (S)-1-(4,4,6,6,6-PENTADEUTERO-5-HYDROXYHEXYL)-3-7-DIMETHYL-1H-PURINE-2,6(3H,7H)-DIONE

(71) Applicants: Joanna A. Bis, Cary, NC (US); David H. Igo, Raleigh, NC (US); David J. Turnquist, Winchester, MA (US)

(72) Inventors: Joanna A. Bis, Cary, NC (US); David H. Igo, Raleigh, NC (US); David J. Turnquist, Winchester, MA (US)

(73) Assignee: CONCERT PHARMACEUTICALS, INC., Lexington, MA (US)

(21) Appl. No.: 13/820,022

(22) PCT Filed: Sep. 1, 2011
(86) PCT No.: PCT/US11/50229

§ 371 Date: Aug. 9, 2013

Related U.S. Application Data
(60) Provisional application No. 61/379,179, filed on Sep. 1, 2010.

(51) Int. Cl. C07D 473/10

(52) U.S. Cl. CPC C07D 473/10
USPC 514/263.36; 544/267

(57) ABSTRACT

The present invention provides individual crystalline polymorphs of (S)-1-(4,4,6, 6,6-pentadeutero-5-hydroxy-hexyl)-3,7-dimethyl-1H-purine-2,6(3H,7H)-dione designated Form 1, Form 2, Form 3 and Form 4. Each polymorph disclosed herein is characterized according to one or more of (a) powder X-ray diffraction data (“XRPD”); (b) differential scanning calorimetry (“DSC”); and (e) thermo gravimetric analysis (TGA).

### RELATED APPLICATION

This application claims the benefit of U.S. Provisional Application No. 61/379,179, filed on Sep. 1, 2010. The entire teachings of the above application is incorporated herein by reference.

### BACKGROUND OF THE INVENTION

The compound (S)-1-(4,4,6,6,6-pentadeutero-5-hydroxyhexyl)-3,7-dimethyl-1H-purine-2,6(3H,7H)-dione is a deuterated metabolite of pentoxifylline, a methylxanthine derivative with complex properties including hemorrheologic and anti-inflammatory effects. It is Compound 121(S) described in U.S. patent application No. 61/239,342 on page 27, lines 1-5, which are incorporated by reference herein, and has the Formula I:

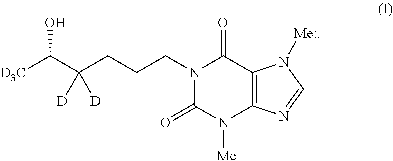

It is well known that the crystalline polymorph form of a particular drug is often an important determinant of the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorph form. In cases where two or more polymorph substances can be produced, it is desirable to have a method to make both polymorphs in pure form. In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc are deemed to be critical. In other situations, a different polymorph may be preferred for greater solubility and/or superior pharmacokinetics.

Because improved drug formulations, showing, for example, better bioavailability or better stability are consistently sought, there is an ongoing need for new or purer polymorphic forms of existing drug molecules. The various crystalline polymorphs of (S)-1-(4,4,6,6,6-pentadeutero-5-hydroxyhexyl)-3,7-dimethyl-1H-purine-2,6(3H,7H)-dione described herein helps meet these and other needs.

### BRIEF DESCRIPTION OF THE DRAWINGS

FIG. 1 depicts the normalized powder X-ray diffraction pattern of Form 1 of (S)-1-(4,4,6,6,6-pentadeutero-5-hydroxyhexyl)-3,7-dimethyl-1H-purine-2,6(3H,7H)-dione with the diffraction angles from 0 to 40 degrees.

FIG. 2 depicts the differential scanning calorimetry (“DSC”) thermogram of Form 1 of (S)-1-(4,4,6,6,6-pentadeutero-5-hydroxyhexyl)-3,7-dimethyl-1H-purine-2,6(3H,7H)-dione.

FIG. 3 depicts the FT-Raman spectrum of Form 1 of (S)-1-(4,4,6,6,6-pentadeutero-5-hydroxyhexyl)-3,7-dimethyl-1H-purine-2,6(3H,7H)-dione.

FIG. 4 depicts the FT-IR spectrum of Form 1 of (S)-1-(4,4,6,6,6-pentadeutero-5-hydroxyhexyl)-3,7-dimethyl-1H-purine-2,6(3H,7H)-dione.

FIG. 5 depicts the normalized powder X-ray diffraction pattern of Form 2 of (S)-1-(4,4,6,6,6-pentadeutero-5-hydroxyhexyl)-3,7-dimethyl-1H-purine-2,6(3H,7H)-dione with the diffraction angles from 0 to 40 degrees.

FIG. 6 depicts the differential scanning calorimetry (“DSC”) thermogram of Form 2 of (S)-1-(4,4,6,6,6-pentadeutero-5-hydroxyhexyl)-3,7-dimethyl-1H-purine-2,6(3H,7H)-dione.

FIG. 7 depicts the normalized powder X-ray diffraction pattern of Form 3 of (S)-1-(4,4,6,6,6-pentadeutero-5-hydroxyhexyl)-3,7-dimethyl-1H-purine-2,6(3H,7H)-dione with the diffraction angles from 0 to 40 degrees.

FIG. 8 depicts the differential scanning calorimetry (“DSC”) thermogram of Form 3 of (S)-1-(4,4,6,6,6-pentadeutero-5-hydroxyhexyl)-3,7-dimethyl-1H-purine-2,6(3H,7H)-dione.

FIG. 9 depicts the normalized powder X-ray diffraction pattern of Form 4 of (S)-1-(4,4,6,6,6-pentadeutero-5-hydroxyhexyl)-3,7-dimethyl-1H-purine-2,6(3H,7H)-dione with the diffraction angles from 0 to 40 degrees.

FIG. 10 depicts the differential scanning calorimetry (“DSC”) thermogram of Form 4 of (S)-1-(4,4,6,6,6-pentadeutero-5-hydroxyhexyl)-3,7-dimethyl-1H-purine-2,6(3H,7H)-dione.

### SUMMARY OF THE INVENTION

The present invention provides crystalline polymorphs of optionally deuterated (S)-1-(5-hydroxyhexyl)-3,7-dimethyl-1H-purine-2,6(3H,7H)-dione having one or more of the (i) powder X-ray diffraction peaks, (ii) DSC endotherms, (iii) FT-Raman spectral characteristics, (iv) FT-IR spectral characteristics, and (v) thermogravimetric characteristics that are disclosed herein for (S)-1-(4,4,6,6,6-pentadeutero-5-hydroxyhexyl)-3,7-dimethyl-1H-purine-2,6(3H,7H)-dione. In one embodiment. In one embodiment, the present invention provides crystalline polymorphs of (S)-1-(4,4,6,6,6-pentadeutero-5-hydroxyhexyl)-3,7-dimethyl-1H-purine-2,6(3H,7H)-dione designated Form 1, Form 2, Form 3 and Form 4. Each polymorph disclosed herein is characterized according to one or more of (a) powder X-ray diffraction data (“XRPD”); (b) differential scanning calorimetry (“DSC”); (c) FT-Raman spectroscopy; (d) FT-infrared spectroscopy; and (e) thermogravimetric analysis (TGA).

In one embodiment, the invention is directed to the Form 1, Form 2, Form 3 or Form 4 polymorph. In one aspect of this embodiment, the Form 1 polymorph is substantially free of other forms of (S)-1-(4,4,6,6,6-pentadeutero-5-hydroxyhexyl)-3,7-dimethyl-1H-purine-2,6(3H,7H)-dione. Here “other forms” includes other crystalline forms as well as (S)-1-(4,4,6,6,6-pentadeutero-5-hydroxyhexyl)-3,7-dimethyl-1H-purine-2,6(3H,7H)-dione in amorphous form. In one aspect of this embodiment, the Form 1 polymorph is substantially free of the other three forms disclosed herein. In this aspect, the term “substantially free of other forms” means that the sum of the amounts of other forms of is less than 50%, more preferably equal to or less than 20%, more preferably equal to or less than 10%, more preferably equal to or less than 5%, more preferably equal to or less than 1%, or more preferably equal to or less than 0.1%, of the amount of the Form 1 polymorph.

The present invention further provides compositions comprising the Form 1, Form 2, Form 3 or Form 4 polymorph of (S)-1-(4,4,6,6,6-pentadeutero-5-hydroxyhexyl)-3,7-dimethyl-1H-purine-2,6(3H,7H)-dione. In one embodiment, such compositions are pharmaceutically acceptable compositions additionally comprising a pharmaceutically acceptable carrier.

The present invention further provides a method of treating a mammal having a disease or syndrome that is beneficially treated by pentoxifylline comprising administering to the mammal a therapeutically effective amount of the Form 1, Form 2, Form 3 or Form 4 polymorph of (S)-1-(4,4,6,6,6-pentadeutero-5-hydroxyhexyl)-3,7-dimethyl-1H-purine-2,6(3H,7H)-dione.

The present invention further provides a method of treating a mammal suffering from an indication disclosed herein, comprising administering to said mammal a therapeutically effective amount of the Form 1, Form 2, Form 3 or Form 4 polymorph of (S)-1-(4,4,6,6,6-pentadeutero-5-hydroxyhexyl)-3,7-dimethyl-1H-purine-2,6(3H,7H)-dione. In one embodiment, the indication is diabetic nephropathy.

The present invention further provides a method of synthesizing the Form 1, Form 2, Form 3 or Form 4 polymorph of (S)-1-(4,4,6,6,6-pentadeutero-5-hydroxyhexyl)-3,7-dimethyl-1H-purine-2,6(3H,7H)-dione by performing hydrogen-deuterium exchange on (S)-1-(4,4,6,6,6-pentadeutero-5-hydroxyhexyl)-3,7-dimethyl-8-deutero-1H-purine-2,6(3H,7H)-dione.

The present invention further provides the Form 1, Form 2, Form 3 or Form 4 polymorph of (S)-1-(4,4,6,6,6-pentadeutero-5-hydroxyhexyl)-3,7-dimethyl-1H-purine-2,6(3H,7H)-dione prepared by any of the methods described herein.

### DEFINITIONS

The term “Form 1 of (S)-1-(4,4,6,6,6-pentadeutero-5-hydroxyhexyl)-3,7-dimethyl-1H-purine-2,6(3H,7H)-dione” refers to the Form 1 crystalline polymorph of (S)-1-(4,4,6,6,6-pentadeutero-5-hydroxyhexyl)-3,7-dimethyl-1H-purine-2,6(3H,7H)-dione. The terms “Form 1 of (S)-1-(4,4,6,6,6-pentadeutero-5-hydroxyhexyl)-3,7-dimethyl-1H-purine-2,6(3H,7H)-dione”, “Form 1”, and “the Form 1 polymorph” are used interchangeably herein.

When the term “(S)-1-(4,4,6,6,6-pentadeutero-5-hydroxyhexyl)-3,7-dimethyl-1H-purine-2,6(3H,7H)-dione” is used without specifying the crystalline form (such as Form 1, Form 2, and so on), this term refers to the compound in any form, such as crystalline, amorphous, or other, or in a combination of forms.

Throughout this application, unless otherwise specified, when a particular position is designated as having deuterium, it is understood that the abundance of deuterium at that position has a minimum isotopic enrichment factor of at least 3340 (50.1% deuterium incorporation) at each atom designated as deuterium in said compound.”) Preferably, the %age of deuterium incorporation is at least 80%, at least 85%, at least 90%, at least 95%, at least 98%, or at least 99%.

Experimental

### XRPD

X-ray powder diffraction (XRPD) data were obtained using a PANalytical X'Pert Pro diffractometer equipped with an X'Celerator detector. The sample was flattened on a zero-background silicon holder and was run immediately after preparation under ambient conditions. A continuous 2-theta scan range of 2° to 40° was used with a Cu Kα (1.5406 Å) radiation source and a generator power of 45 kV and 40 mA. A step size of 0.0167 degrees per 2-theta step was used and the sample was rotated at 30 rpm.

### Thermal Analysis

DSC thermograms were recorded on a TA Instruments Q 1000 Differential Scanning calorimeter. The sample was weighed into an aluminium pan, a pan lid placed on top and lightly crimped without sealing the pan. The experiments were conducted using a heating rate of 15° C./min.

TGA thermograms were recorded on a TA Instruments Q5000 Themrogravimetric Analyzer. The sample was weighed into an aluminum pan, and experiments were conducted using a heating rate of 15° C./min.

### FT-IR

FT-IR spectra were recorded on a Nicolet 6700 FTIR instrument equipped with a SensIR Durascope Diamond Attenuated Total Reflectance (DATR) accessory. A background scan was collected with no sample on the accessory. Sample data was collected after a small sample (˜2 mg) was pressed against the diamond window. Data was acquired at a resolution of 4 cm⁻¹.

### DETAILED DESCRIPTION OF THE INVENTION

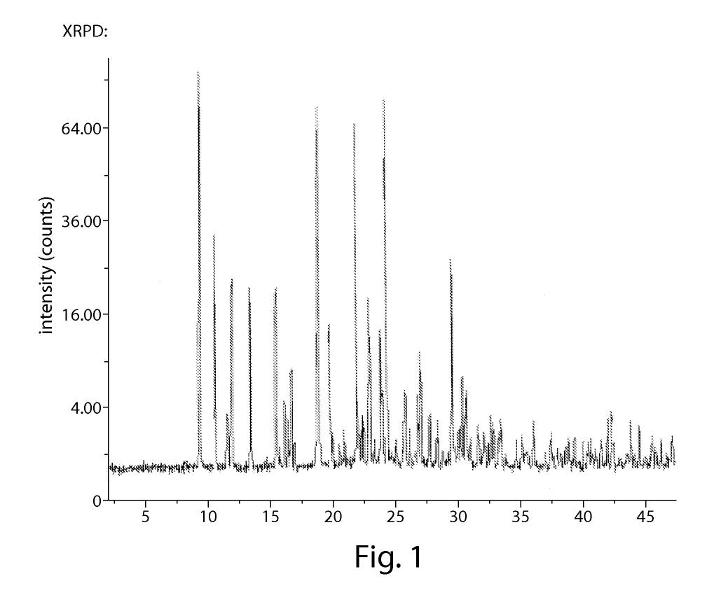

The present invention provides in one embodiment a crystalline polymorph of (S)-1-(4,4,6,6,6-pentadeutero-5-hydroxyhexyl)-3,7-dimethyl-1H-purine-2,6(3H,7H)-dione, referred to herein as Form 1. Form 1 is an anhydrous, non-solvated crystalline form. Form 1 can be described by one or more solid state analytical methods, for example, by its powder X-ray diffraction pattern which is provided in FIG. 1. Powder X-ray diffraction 2-theta values characteristic for Form 1 are provided in Table 1 below.

TABLE 1
2-theta Peak Values and intensities of Form 1 polymorph of
(S)-1-(4,4,6,6,6-pentadeutero-5-hydroxyhexyl)-3,7-dimethyl-
1H-purine-2,6(3H,7H)-dione.

| [°2-Theta] | Height [cts] |
|---|---|
| 9.3 | 8098.5 |
| 10.5 | 3053.6 |
| 11.9 | 2231.7 |
| 13.4 | 2021.2 |
| 15.5 | 1950.3 |
| 16.6 | 737.8 |
| 18.7 | 1234.7 |
| 18.8 | 7119.6 |
| 19.7 | 1424.6 |
| 21.8 | 6396.0 |
| 22.9 | 1766.9 |
| 23.8 | 1277.7 |
| 24.3 | 7167.3 |
| 27.0 | 919.8 |
| 29.5 | 2667.3 |

In some embodiments, Form 1 is characterized as having a powder X-ray diffraction pattern having two or more characteristic peaks, in terms of 2-theta, selected from 9.3, 13.4, 18.8, 19.7, 21.8, 22.9, 23.8, 29.5 degrees, at ambient temperature. In one aspect of this embodiment, Form 1 is characterized by the peaks at 2-theta values of 9.3, 18.8, 21.8 and 24.3 degrees. In one aspect of this embodiment, Form 1 is characterized as having a powder X-ray diffraction pattern peaks, in terms of 2-theta, at each of 9.3, 13.4, 18.8, 19.7, 21.8, 22.9, 23.8, and 29.5 degrees, at ambient temperature. In still other aspects, Form 1 is characterized by 2-theta peaks at each of 9.3, 10.5, 11.9, 13.4, 15.5, 16.6, 18.7, 18.8, 19.7, 21.8, 22.9, 23.8, 24.3, 27.0, and 29.5 degrees, at ambient temperature. In yet further aspects, Form 1 is characterized by a powder X-ray diffraction pattern substantially as shown in FIG. 1, at ambient temperature. The relative intensities of the peaks can vary, depending upon the sample preparation technique, the sample mounting procedure, the particular instrument employed, and the morphology of the sample. Moreover, instrument variation and other factors can affect the 2-theta values. Therefore, the XRPD peak assignments for Form 1 and all other crystalline forms disclosed herein, can vary by ±0.2°.

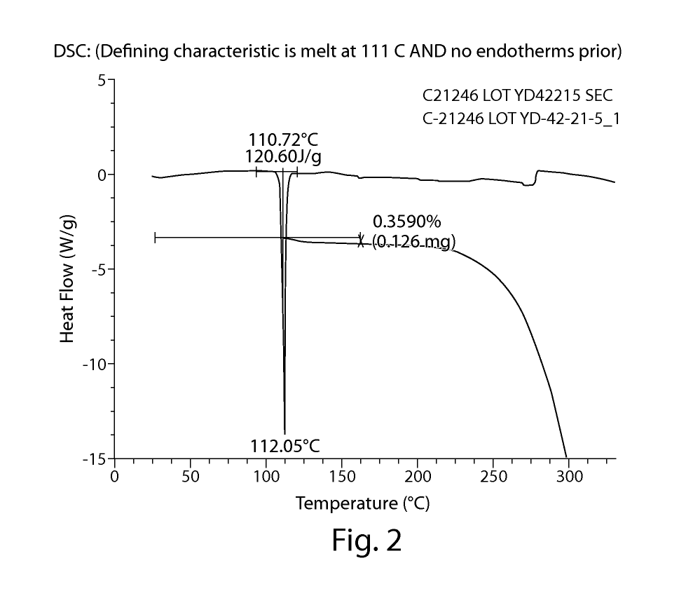

In another embodiment, Form 1 is identified by its characteristic melting point of 111° C. (onset value). In one aspect of this embodiment, Form 1 is characterized by a DSC thermogram showing a maximum at 110.7° C. (onset value). In a related aspect, Form 1 is identified by the differential calorimetric scanning (DSC) thermogram as shown in FIG. 2. For DSC, it is known that the temperatures observed will depend upon the rate of temperature change as well as sample preparation technique and the particular instrument employed. Thus, the values reported herein for Form 1 and all other crystalline forms relating to melting point and DSC thermograms can vary by ±4° C.

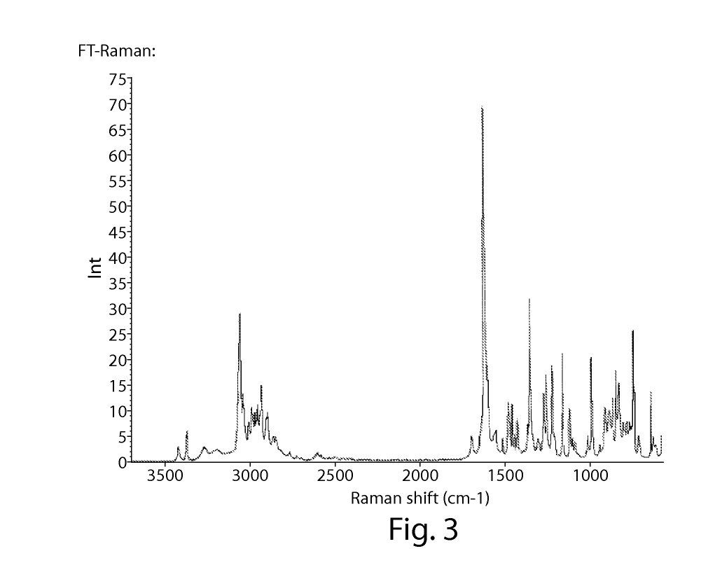

In another embodiment, Form 1 is identified by the FT-Raman spectrum shown in FIG. 3.

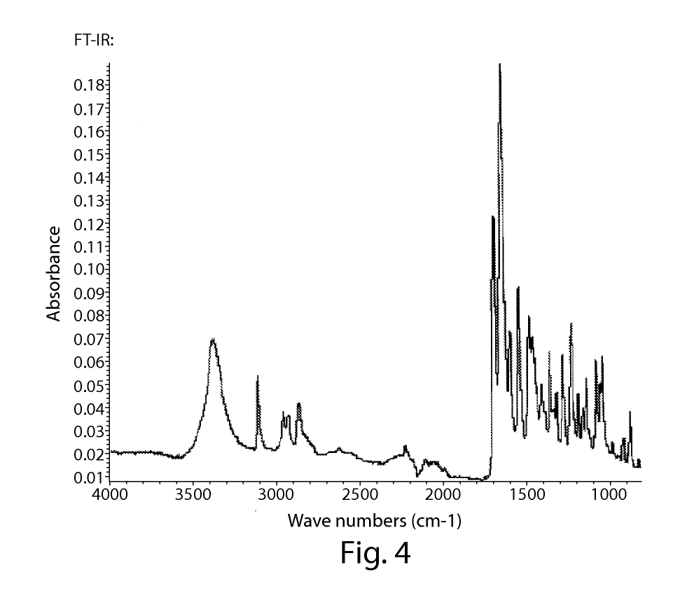

In another embodiment, Form 1 is identified by the FT-IR spectrum shown in FIG. 4. The pattern shows characteristic IR shift peaks at 615, 751, 761, 881, 1043, 1076, 1137, 1162, 1186, 1228, 1284, 1321, 1359, 1409, 1484, 1547, 1602, 1652, 1695, 2871, 2961, 3112, and 3379 cm-1.

Form 1 is more thermodynamically stable than any of Forms 2, 3 and 4. Forms 2, 3 and 4 each convert to Form 1 upon air drying, storage and/or slurrying at room temperature.

In one embodiment, the Form 1 polymorph of (S)-1-(4,4,6,6,6-pentadeutero-5-hydroxyhexyl)-3,7-dimethyl-1H-purine-2,6(3H,7H)-dione has at least 98% deuterium incorporation at each position designated as deuterium in Formula I as determined by ¹H-NMR. In one aspect of this embodiment, the Form 1 polymorph of (S)-1-(4,4,6,6,6-pentadeutero-5-hydroxyhexyl)-3,7-dimethyl-1H-purine-2,6(3H,7H)-dione is substantially free of (S)-1-(4,4,6,6,6-pentadeutero-5-hydroxyhexyl)-3,7-dimethyl-8-deutero-1H-purine-2,6(3H,7H)-dione as determined by ¹H-NMR. In this aspect, the term “substantially free of (S)-1-(4,4,6,6,6-pentadeutero-5-hydroxyhexyl)-3,7-dimethyl-8-deutero-1H-purine-2,6(3H,7H)-dione” means that the amount of (S)-1-(4,4,6,6,6-pentadeutero-5-hydroxyhexyl)-3,7-dimethyl-8-deutero-1H-purine-2,6(3H,7H)-dione is equal to or less than 5%, more preferably equal to or less than 1%, or more preferably equal to or less than 0.1%, of the amount of the Form 1 polymorph.

The invention is also directed to a process for the preparation of the Form 1 polymorph, comprising (i) forming a slurry of (S)-1-(4,4,6,6,6-pentadeutero-5-hydroxyhexyl)-3,7-dimethyl-1H-purine-2,6(3H,7H)-dione in ethyl acetate and n-heptane, and (ii) cooling the slurry to a temperature sufficiently low to form the Form 1 polymorph. In one embodiment, the volume ratio of ethyl acetate to n-heptane in the slurry is 5.5. In one aspect of this embodiment, the slurry is formed at a temperature of 60° C. In one aspect, the slurry is cooled to 20° C. In a more particular aspect, the Form 1 polymorph is formed after the slurry is cooled to 20° C., then filtered and washed with n-heptane.

In one embodiment, the Form 1 polymorph is prepared in a three-step process beginning with commercially available pentoxifylline as detailed in the Example section.

The invention is also directed to a process for the preparation of the Form 1 polymorph, comprising (i) dissolving (S)-1-(4,4,6,6,6-pentadeutero-5-hydroxyhexyl)-3,7-dimethyl-1H-purine-2,6(3H,7H)-dione in a solvent selected from ethanol, ethyl acetate, and acetone, and (ii) slowly evaporating the solvent to form the Form 1 polymorph. Slowly evaporating the solvent may be achieved, for example, by allowing the dissolved (S)-1-(4,4,6,6,6-pentadeutero-5-hydroxyhexyl)-3,7-dimethyl-1H-purine-2,6(3H,7H)-dione to stand at ambient temperature and evaporating the solvent without supplying external heat. In one embodiment the evaporating occurs over 2-28 days at ambient temperature, preferably from a saturated solution.

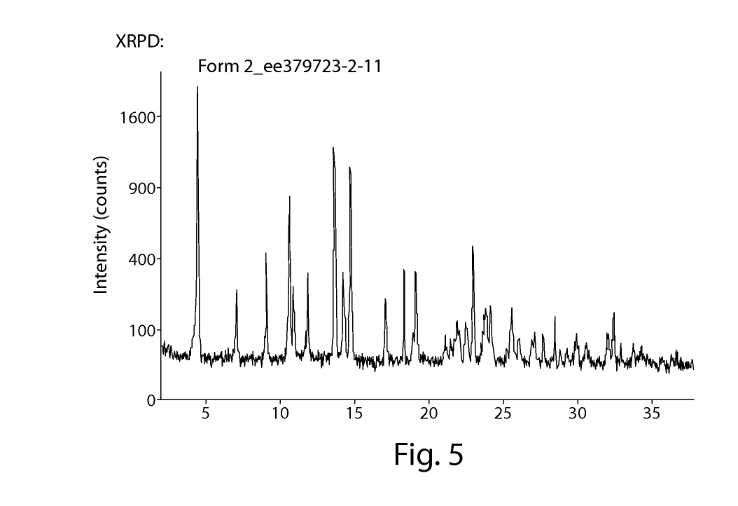

In another embodiment the present invention provides an anhydrous, non-solvated crystalline polymorph of (S)-1-(4,4,6,6,6-pentadeutero-5-hydroxyhexyl)-3,7-dimethyl-1H-purine-2,6(3H,7H)-dione, referred to herein as Form 2. In one aspect, Form 2 is identified by its powder X-ray diffraction pattern which is provided in FIG. 5. Powder X-ray diffraction 2-theta values characteristic for Form 2 are provided in Table 2 below.

TABLE 2
2-theta Peak Values and intensities of Form 2 polymorph of
(S)-1-(4,4,6,6,6-pentadeutero-5-hydroxyhexyl)-3,7-dimethyl-
1H-purine-2,6(3H,7H)-dione.

| [°2-Theta] | Height [cts] |
|---|---|
| 4.5 | 2231.7 |
| 7.1 | 252.4 |
| 9.1 | 469.2 |
| 10.7 | 943.5 |
| 10.9 | 275.2 |
| 11.9 | 369.2 |
| 13.7 | 1532.1 |
| 14.3 | 352.8 |
| 14.8 | 1312.8 |
| 17.1 | 204.7 |
| 18.4 | 382.3 |
| 19.2 | 366.5 |
| 23.0 | 518.5 |

In some embodiments, the Form 2 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern having two or more characteristic peaks, in terms of 2-theta, selected from 4.5, 9.1, 10.7, 13.7, 14.1, 14.8, 18.4, 19.2, and 23.0 degrees at ambient temperature. In one aspect of this embodiment, Form 2 is characterized by the peaks at 2-theta values of 4.5, 13.7, and 14.8 degrees. In one aspect, the Form 2 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern peaks, in terms of 2-theta, at each of 4.5, 9.1, 10.7, 13.7, 14.3, 14.8, 18.4, 19.2, and 23.0 degrees at ambient temperature. In still other aspects, Form 2 is characterized by 2-theta peaks at each of 4.5, 7.1, 9.1, 10.7, 10.9, 11.9, 13.7, 14.3, 14.8, 17.1, 18.4, 19.2, and 23.0 degrees at ambient temperature. In yet further aspects, Form 2 is characterized by a powder X-ray diffraction pattern substantially as shown in FIG. 5 at ambient temperature.

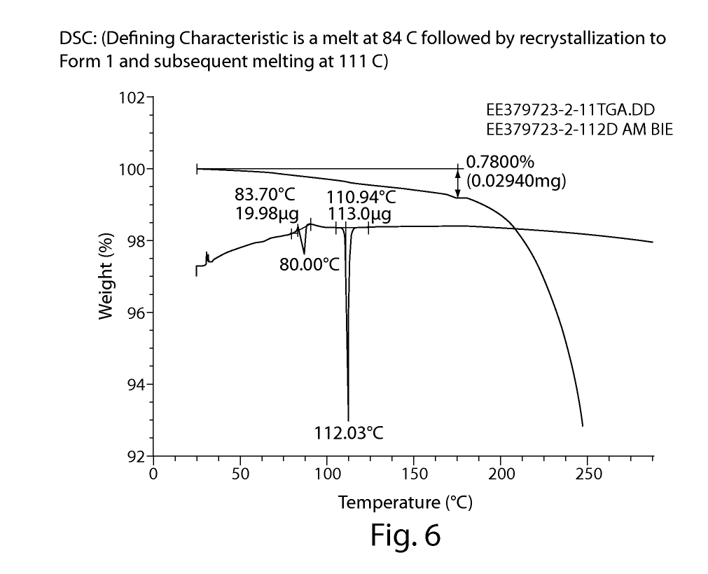

In one embodiment, Form 2 is identified by a characteristic thermal event at 84° C. (onset value). In one aspect of this embodiment, Form 2 is characterized by a DSC thermogram showing a first endothermic event at 84° C. (onset value). This is believed to be the temperature at which Form 2 is converted to Form 1. In another aspect, Form 2 is characterized by a DSC thermogram showing a first endothermic event at 84° C. (onset value) and a second endothermic event at 111° C. In still another aspect, Form 2 may be identified by the differential calorimetric scanning (DSC) thermogram as shown in FIG. 6.

In one embodiment, the Form 2 polymorph of (S)-1-(4,4,6,6,6-pentadeutero-5-hydroxyhexyl)-3,7-dimethyl-1H-purine-2,6(3H,7H)-dione has at least 98% deuterium incorporation at each position designated as deuterium in Formula I as determined by ¹H-NMR. In one aspect of this embodiment, the Form 2 polymorph of (S)-1-(4,4,6,6,6-pentadeutero-5-hydroxyhexyl)-3,7-dimethyl-1H-purine-2,6(3H,7H)-dione is substantially free of (S)-1-(4,4,6,6,6-pentadeutero-5-hydroxyhexyl)-3,7-dimethyl-8-deutero-1H-purine-2,6(3H,7H)-dione as determined by ¹H-NMR. In this aspect, the term “substantially free of (S)-1-(4,4,6,6,6-pentadeutero-5-hydroxyhexyl)-3,7-dimethyl-8-deutero-1H-purine-2,6(3H,7H)-dione” means that the amount of (S)-1-(4,4,6,6,6-pentadeutero-5-hydroxyhexyl)-3,7-dimethyl-8-deutero-1H-purine-2,6(3H,7H)-dione is equal to or less than 5%, more preferably equal to or less than 1%, or more preferably equal to or less than 0.1%, of the amount of the Form 2 polymorph.

The Form 2 polymorph may be prepared from Form 1 by various solution-phase methods including rapid solvent removal (e.g., water, isopropyl acetate, or toluene), cooling (isopropyl acetate and toluene), and lyophilization (acetonitrile/water 5:2).

In another embodiment, Form 2 is identified by characteristic IR shift peaks at 615, 750, 763, 1016, 1038, 1126, 1152, 1187, 1230, 1260, 1287, 1323, 1358, 1413, 1460, 1486, 1550, 1604, 1647, 1702, 2960, 3121, 3392, 3464 cm⁻¹

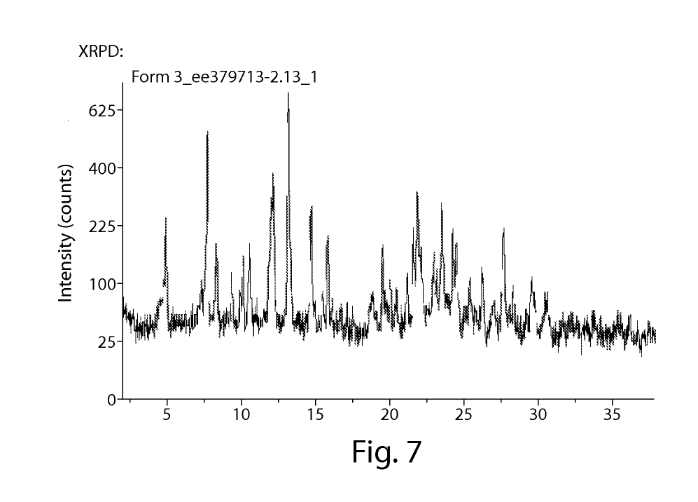

In another embodiment the present invention provides an anhydrous, non-solvated crystalline polymorph of (S)-1-(4,4,6,6,6-pentadeutero-5-hydroxyhexyl)-3,7-dimethyl-1H-purine-2,6(3H,7H)-dione, referred to herein as Form 3. In one aspect, Form 3 is identified by its powder X-ray diffraction pattern which is provided in FIG. 7. Form 3 has not been isolated as phase-pure crystalline form. Powder X-ray diffraction 2-theta values characteristic for Form 3 are provided in Table 3 below.

TABLE 3
2-theta Peak Values of Form 3 polymorph of (S)-1-
(4,4,6,6,6-pentadeutero-5-hydroxyhexyl)-3,7-dimethyl-
1H-purine-2,6(3H,7H)-dione.

| [°2-Theta] | Height [cts] |
|---|---|
| 4.9 | 1078.1 |
| 7.7 | 2537.9 |
| 8.3 | 661.0 |
| 10.1 | 637.8 |
| 12.1 | 1492.8 |
| 13.2 | 3309.9 |
| 14.7 | 1244.4 |
| 15.8 | 862.8 |
| 19.5 | 727.8 |
| 21.6 | 846.7 |
| 23.5 | 1088.1 |
| 24.4 | 768.0 |
| 27.7 | 935.2 |

In some embodiments, the Form 3 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern having two or more characteristic peaks, in terms of 2-theta, selected from 4.9, 7.7, 8.3, 13.2, 14.7, 15.8, 19.5, 21.6, 23.5, 27.7 degrees at ambient temperature. In one aspect of this embodiment, Form 3 is characterized by the peaks at 2-theta values of 7.7 and 13.2 degrees. In one aspect, the Form 3 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern peaks, in terms of 2-theta, at each of 4.9, 7.7, 8.3, 13.2, 14.7, 15.8, 19.5, 21.6, 23.5, 27.7 degrees at ambient temperature. In still other aspects, Form 3 is characterized by 2-thetapeaks at each of 4.9, 7.7, 8.3, 10.1, 12.1, 13.2, 14.7, 15.8, 19.5, 21.6, 23.5, 24.2, 27.7 degrees at ambient temperature. In yet further aspects, Form 3 is characterized by a powder X-ray diffraction pattern substantially as shown in FIG. 7 at ambient temperature.

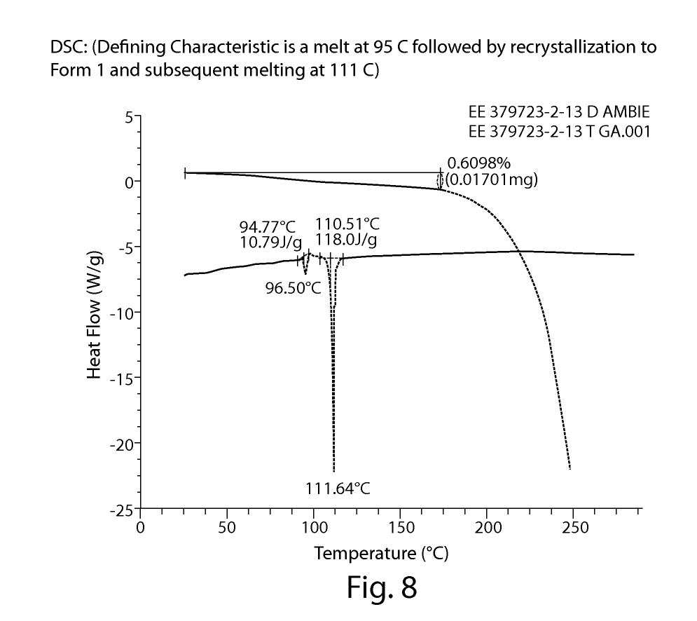

In one embodiment, Form 3 is identified by a characteristic thermal event at 95° C. (onset value). In one aspect of this embodiment, Form 3 is characterized by a DSC thermogram showing a first endothermic event at 95° C. (onset value). This is believed to be the temperature at which Form 3 is converted to Form 1. In another aspect, Form 3 is characterized by a DSC thermogram showing a first endothermic event at 95° C. (on-set value) and a second endothermic event at 111° C. In a related embodiment, Form 3 may be identified by the differential calorimetric scanning (DSC) thermogram as shown in FIG. 8.

In one embodiment, the Form 3 polymorph of (S)-1-(4,4,6,6,6-pentadeutero-5-hydroxyhexyl)-3,7-dimethyl-1H-purine-2,6(3H,7H)-dione has at least 98% deuterium incorporation at each position designated as deuterium in Formula I as determined by ¹H-NMR. In one aspect of this embodiment, the Form 3 polymorph of (S)-1-(4,4,6,6,6-pentadeutero-5-hydroxyhexyl)-3,7-dimethyl-1H-purine-2,6(3H,7H)-dione is substantially free of (S)-1-(4,4,6,6,6-pentadeutero-5-hydroxyhexyl)-3,7-dimethyl-8-deutero-1H-purine-2,6(3H,7H)-dione as determined by ¹H-NMR. In this aspect, the term “substantially free of (S)-1-(4,4,6,6,6-pentadeutero-5-hydroxyhexyl)-3,7-dimethyl-8-deutero-1H-purine-2,6(3H,7H)-dione” means that the amount of (S)-1-(4,4,6,6,6-pentadeutero-5-hydroxyhexyl)-3,7-dimethyl-8-deutero-1H-purine-2,6(3H,7H)-dione is equal to or less than 5%, more preferably equal to or less than 1%, or more preferably equal to or less than 0.1%, of the amount of the Form 3 polymorph.

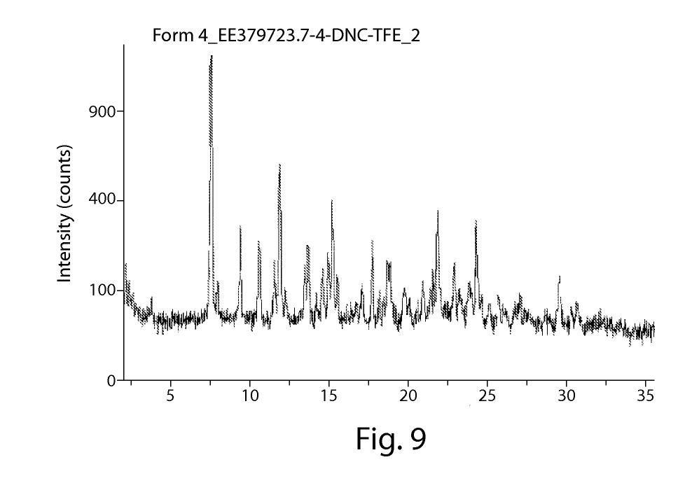

The Form 3 polymorph of this invention may be prepared from the Form 1 polymorph by various evaporative methods that involved rapid removal of solvent (e.g. isopropyl acetate, acetonitrile, and/or toluene). In another embodiment the present invention provides an anhydrous, non-solvated crystalline polymorph of (S)-1-(4,4,6,6,6-pentadeutero-5-hydroxyhexyl)-3,7-dimethyl-1H-purine-2,6(3H,7H)-dione, referred to herein as Form 4. In one aspect, Form 4 is identified by its powder X-ray diffraction pattern which is provided in FIG. 9. Powder X-ray diffraction 2-theta values characteristic for Form 4 are provided in Table 3 below.

TABLE 4
2-theta Peak Values of Form 4 polymorph of(S)-1-
(4,4,6,6,6-pentadeutero-5-hydroxyhexyl)-3,7-dimethyl-
1H-purine-2,6(3H,7H)-dione.

| [°2-Theta] | Height [cts] |
|---|---|
| 7.5 | 6261.5 |
| 15.1 | 1859.8 |
| 17.7 | 886.0 |

In some embodiments, the Form 4 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern having two or more characteristic peaks, in terms of 2-theta, selected from 7.5, 15.1 and 17.7 degrees at ambient temperature. In one aspect, the Form 4 polymorph of (S)-1-(4,4,6,6,6-pentadeutero-5-hydroxyhexyl)-3,7-dimethyl-1H-purine-2,6(3H,7H)-dione is characterized as having a powder X-ray diffraction pattern peaks, in terms of 2-theta, at each of 7.5, 15.1 and 17.7 degrees at ambient temperature. In still other aspects, Form 4 is characterized by a powder X-ray diffraction pattern substantially as shown in FIG. 9 at ambient temperature.

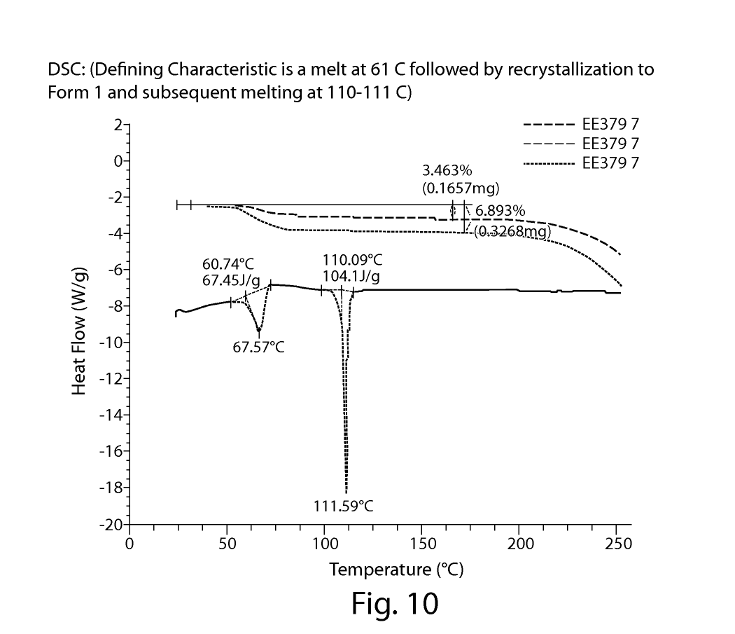

Form 4 can also be identified by a characteristic thermal event at 61° C. (onset value). In one aspect of this embodiment, Form 4 is characterized by a DSC thermogram showing a first endothermic event at 61° C. (onset value). In another aspect, Form 4 is characterized by a DSC thermogram showing a first endothermic event at 61° C. (onset value) and a second endothermic event at 111° C. In a related embodiment, Form 4 may be identified by the differential calorimetric scanning (DSC) thermogram as shown in FIG. 10.

In one embodiment, the Form 4 polymorph of (S)-1-(4,4,6,6,6-pentadeutero-5-hydroxyhexyl)-3,7-dimethyl-1H-purine-2,6(3H,7H)-dione has at least 98% deuterium incorporation at each position designated as deuterium in Formula I as determined by ¹H-NMR. In one aspect of this embodiment, the Form 4 polymorph of (S)-1-(4,4,6,6,6-pentadeutero-5-hydroxyhexyl)-3,7-dimethyl-1H-purine-2,6(3H,7H)-dione is substantially free of (S)-1-(4,4,6,6,6-pentadeutero-5-hydroxyhexyl)-3,7-dimethyl-8-deutero-1H-purine-2,6(3H,7H)-dione as determined by ¹H-NMR. In this aspect, the term “substantially free of (S)-1-(4,4,6,6,6-pentadeutero-5-hydroxyhexyl)-3,7-dimethyl-8-deutero-1H-purine-2,6(3H,7H)-dione” means that the amount of (S)-1-(4,4,6,6,6-pentadeutero-5-hydroxyhexyl)-3,7-dimethyl-8-deutero-1H-purine-2,6(3H,7H)-dione is equal to or less than 5%, more preferably equal to or less than 1%, or more preferably equal to or less than 0.1%, of the amount of the Form 4 polymorph.

Form 4 may be prepared from Form 1 using solution phase methods that involved rapid removal of solvent (e.g. acetonitrile) or lyophilization (dimethyl carbonate/trifluoroethanol).

### Compositions

The invention also provides pyrogen-free pharmaceutical compositions comprising an effective amount of the Form 1 polymorph of this invention; and a pharmaceutically acceptable carrier. The carrier(s) are “pharmaceutically acceptable” in the sense of being not deleterious to the recipient thereof in an amount used in the medicament.

In certain embodiments, the ratio of Form 1 to (Form 2+Form 3+Form 4) in such pharmaceutical compositions is greater than 50:50, equal to or greater than 80:20, equal to or greater than 90:10, equal to or greater than 95:5, equal to or greater than 99:1; or 100:0.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

In certain embodiments, the compositions are extended release oral formulations. In one aspect of this embodiment, the controlled release formulation will be based on a diffusion-controlled hydrogel tablet. In a more specific aspect of this embodiment, the controlled release formulation comprises high molecular weight HPMC polymer. In an even more specific aspect, the high molecular weight HPMC polymer is HPMC K15M CR. In another even more specific aspect, the high molecular weight HPMC polymer comprises between 30 and 70% (w/w) of the composition.

In another embodiment, the Form 1 polymorph comprises between 28 and 68% (w/w) of the composition. In this embodiment, magnesium stearate and microcrystalline cellulose comprise about 2% (w/w) of the composition.

### Methods of Treatment

According to another embodiment, the invention provides a method of treating a disease in a patient in need thereof that is beneficially treated by pentoxifylline comprising the step of administering to said patient an effective amount of a polymorphic form disclosed herein, such as Form 1 polymorph of (S)-1-(4,4,6,6,6-pentadeutero-5-hydroxyhexyl)-3,7-dimethyl-1H-purine-2,6(3H,7H)-dione or a pharmaceutical composition comprising Form 1 polymorph of (S)-1-(4,4,6,6,6-pentadeutero-5-hydroxyhexyl)-3,7-dimethyl-1H-purine-2,6(3H,7H)-dione and a pharmaceutically acceptable carrier.

Such diseases are well known in the art and are disclosed in, but not limited to the following patents and published applications: WO 1988004928, EP 0493682, U.S. Pat. No. 5,112,827, EP 0484785, WO 1997019686, WO 2003013568, WO 2001032156, WO 1992007566, WO 1998055110, WO 2005023193, U.S. Pat. No. 4,975,432, WO 1993018770, EP 0490181, and WO 1996005836. Such diseases include, but are not limited to, peripheral obstructive vascular disease; glomerulonephritis; nephrotic syndrome; nonalcoholic steatohepatitis; Leishmaniasis; cirrhosis; liver failure; Duchenne's muscular dystrophy; late radiation induced injuries; radiation induced lymphedema; radiation-associated necrosis; alcoholic hepatitis; radiation-associated fibrosis; necrotizing enterocolitis in premature neonates; diabetic nephropathy, hypertension-induced renal failure, and other chronic kidney disease; Focal Segmental Glomerulosclerosis; pulmonary sarcoidosis; recurrent aphthous stomatitis; chronic breast pain in breast cancer patients; brain and central nervous system tumors; malnutrition-inflammation-cachexia syndrome; interleukin-1 mediated disease; graft versus host reaction and other allograft reactions; diet-induced fatty liver conditions, atheromatous lesions, fatty liver degeneration and other diet-induced high fat or alcohol-induced tissue-degenerative conditions; human immunodeficiency virus type 1 (HIV-1) and other human retroviral infections; multiple sclerosis; cancer; fibroproliferative diseases; fungal infection; drug-induced nephrotoxicity; collagenous colitis and other diseases and/or conditions characterized by elevated levels of platelet derived growth factor (PDGF) or other inflammatory cytokines; endometriosis; optic neuropathy and CNS impairments associated with acquired immunodeficiency syndrome (AIDS), immune disorder diseases, or multiple sclerosis; autoimmune disease; upper respiratory viral infection; depression; urinary incontinence; irritable bowel syndrome; septic shock; Alzheimer's Dementia; neuropathic pain; dysuria; retinal or optic nerve damage; peptic ulcer; insulin-dependent diabetes; non-insulin-dependent diabetes; diabetic nephropathy; metabolic syndrome; obesity; insulin resistance; dyslipidemia; pathological glucose tolerance; hypertension; hyperlipidemia; hyperuricemia; gout; hypercoagulability; acute alcoholic hepatitis; olfaction disorders; patent ductus arteriosus; and inflammation or injury associated with neutrophil chemotaxis and/or degranulation.

The Form 1 polymorph of (S)-1-(4,4,6,6,6-pentadeutero-5-hydroxyhexyl)-3,7-dimethyl-1H-purine-2,6(3H,7H)-dione can also be used to control intraocular pressure or to stabilize auto-regulation of cerebral blood flow in subjects who require such control as determined by medical examination.

In one particular embodiment, the method of this invention is used to treat a disease or condition in a patient in need thereof selected from intermittent claudication on the basis of chronic occlusive arterial disease of the limbs and other peripheral obstructive vascular diseases; glomerulonephritis; Focal Segmental Glomerulosclerosis; nephrotic syndrome; nonalcoholic steatohepatitis; Leishmaniasis; cirrhosis; liver failure; Duchenne's muscular dystrophy; late radiation induced injuries; radiation induced lymphedema; alcoholic hepatitis; radiation-induced fibrosis; necrotizing enterocolitis in premature neonates; diabetic nephropathy, hypertension-induced renal failure and other chronic kidney diseases; pulmonary sarcoidosis; recurrent aphthous stomatitis; chronic breast pain in breast cancer patients; brain and central nervous system tumors; obesity; acute alcoholic hepatitis; olfaction disorders; endometriosis-associated infertility; malnutrition-inflammation-cachexia syndrome; and patent ductus arteriosus.

In one embodiment, the method of this invention is used to treat diabetic nephropathy, hypertensive nephropathy or intermittent claudication on the basis of chronic occlusive arterial disease of the limbs. In another particular embodiment, the method of this invention is used to treat a disease or condition in a patient in need thereof selected from intermittent claudication on the basis of chronic occlusive arterial disease of the limbs.

In one embodiment, the method of this invention is used to treat chronic kidney disease. The chronic kidney disease may be selected from glomerulonephritis, focal segmental glomerulosclerosis, nephrotic syndrome, reflux uropathy, or polycystic kidney disease.

In one embodiment, the method of this invention is used to treat chronic disease of the liver. The chronic disease of the liver may be selected from nonalcoholic steatohepatitis, fatty liver degeneration or other diet-induced high fat or alcohol-induced tissue-degenerative conditions, cirrhosis, liver failure, or alcoholic hepatitis.

In one embodiment, the method of this invention is used to a diabetes-related disease or condition. This disease may be selected from insulin resistance, retinopathy, diabetic ulcers, radiation-associated necrosis, acute kidney failure or drug-induced nephrotoxicity.

In one embodiment, the method of this invention is used to treat a patient suffering from cystic fibrosis, including those patients suffering from chronic Pseudomonas bronchitis.

In one embodiment, the method of this invention is used to aid in wound healing. Examples of types of wounds that may be treated include venous ulcers, diabetic ulcers and pressure ulcers.

In another particular embodiment, the method of this invention is used to treat a disease or condition in a patient in need thereof selected from insulin dependent diabetes; non-insulin dependent diabetes; metabolic syndrome; obesity; insulin resistance; dyslipidemia; pathological glucose tolerance; hypertension; hyperlipidemia; hyperuricemia; gout; and hypercoagulability.

In one embodiment, the method of this invention is used to treat a disease or condition in a patient in need thereof wherein the disease or condition is selected from anemia, Graves disease, retinal vein occlusion, lupus nephritis, macular degeneration, myelodysplasia, pruritus of HIV origin, pulmonary hypertension, retinal artery occlusion, intestinal inflammation, ischemic optic neuropathy, acute pancreatitis, sickle cell anemia and beta thalassemia.

In one specific embodiment, the method of this invention is used to treat a disease or condition in a patient in need thereof wherein the disease or condition is diabetic nephropathy.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with pentoxifylline. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a Form 1 polymorph of (S)-1-(4,4,6,6,6-pentadeutero-5-hydroxyhexyl)-3,7-dimethyl-1H-purine-2,6(3H,7H)-dione and a second therapeutic agent for treatment of the following conditions (with the particular second therapeutic agent indicated in parentheses following the indication): late radiation induced injuries (α-tocopherol), radiation-induced fibrosis (α-tocopherol), radiation induced lymphedema (α-tocopherol), chronic breast pain in breast cancer patients (α-tocopherol), type 2 diabetic nephropathy (captopril), malnutrition-inflammation-cachexia syndrome (oral nutritional supplement, such as Nepro; and oral anti-inflammatory module, such as Oxepa); and brain and central nervous system tumors (radiation therapy and hydroxyurea).

The combination therapies of this invention also include co-administering a Form 1 polymorph of (S)-1-(4,4,6,6,6-pentadeutero-5-hydroxyhexyl)-3,7-dimethyl-1H-purine-2,6(3H,7H)-dione and a second therapeutic agent for treatment of insulin dependent diabetes; non-insulin dependent diabetes; metabolic syndrome; obesity; insulin resistance; dyslipidemia; pathological glucose tolerance; hypertension; hyperlipidemia; hyperuricemia; gout; and hypercoagulability.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

### EXAMPLE 1

### Synthesis of (S)-1-(4,4,6,6,6-pentadeutero-5-hydroxyhexyl)-3,7-dimethyl-1H-purine-2,6(3H,7H)-dione Form 1

(S)-1-(4,4,6,6,6-pentadeutero-5-hydroxyhexyl)-3,7-dimethyl-1H-purine-2,6(3H,7H)-dione Form 1 is synthesized according to the description below.

Step 1. Intermediate 11. Pentoxifylline (10; 1 mol equiv) was combined with toluene (20 volumes). To the mixture was added D₂O (1.5 volumes) and potassium carbonate (0.25 equiv) and the mixture was heated to reflux (ca. 87° C.) for 3-4 hrs. The mixture was cooled to 40-50° C. and the aqueous layer was removed. To the remaining toluene solution was added D₂O (1.5 volumes) and potassium carbonate (0.25 equiv) and the mixture was heated to reflux (ca. 87° C.) for 3-4 hrs. The mixture was cooled to 40-50° C. and the aqueous layer was removed. To the remaining toluene solution was added D₂O (1.5 volumes) and potassium carbonate (0.25 equiv) and the mixture was heated to reflux (ca. 87° C.) for 3-4 hrs. The mixture was cooled to 40-50° C. and the aqueous layer was removed. The organic layer was concentrated to ca. 5 volumes below 45° C., was cooled to 20-25° C. and then n-heptane (2 volumes) was added, followed by stirring at 20-25° C. for 30 min. The slurry was filtered and washed with n-heptane, followed by drying in vacuo at 40-50° C. to a constant weight. Yield of 10 was approximately 90%.

Step 2. Intermediate 12. Intermediate 11 (1 mole equiv) was charged to a vessel containing 0.1 M KH₂PO₄ buffer (pH 7.0; 22.5 volumes), and dextrose (1.5 wt % relative to 11). To this mixture was added a solution of NAD (0.6 wt %) in 0.1 M KH₂PO₄ (2.5 volumes), a solution of glucose dehydrogenase GDH (0.1 wt %) in 0.1 M KH₂PO₄ (2.5 volumes) and a solution of the ketoreductase KRED-NADH 101 (1 wt %) in 0.1 M KH₂PO₄ (2.5 volumes). The resulting mixture was stirred at 20-30° C. while maintaining the internal pH at 6-7 by periodic addition of 4N aqueous potassium hydroxide. Sodium chloride was added to the mixture and stirred for 30 min. Ethyl acetate was added to the mixture and stirred for 30 min. The mixture was filtered through a Celite bed and the organic layer was removed. The aqueous layer was extracted with ethyl acetate. The organic layers were combined, concentrated and filtered. The filtrate was concentrated and n-heptane was added at 40-60° C., the resulting slurry was cooled to 20-25° C., aged and filtered. The product was washed with n-heptane and dried in vacuo at 40-50° C. to a constant weight. Yield of 12 was approximately 88%.

Alternative preparation of Intermediate 12: A 3-necked 12-L RB flask equipped with a heating mantle, a J-Kem thermocouple, a mechanical stirrer, and a pH probe was charged with glucose (547.5g, Aldrich lot #088K0039) followed by buffer (9.5 vol, 3.47 L). The reaction mixture was stirred to dissolve all solids. A mixture of 11 (365 g) in buffer (2.92 L) was added and the container was rinsed with buffer (1.28 L). The rinse was added to the reactor. Initially, the reaction mixture was a very thin milky suspension. A solution of KRED-NADH-101 (3.65 g, available from CODEXIS), NAD (2.19 g, available from SPECTRUM), GDH (365 mg, available from CODEXIS) in buffer solution (1.46 L) was charged to the reactor. The container was rinsed with buffer (2×0.91 L) and the rinses were added to the reactor. The reaction mixture was warmed to 20-30° C. and monitored by a pH meter. The reaction mixture turned clear after 30 minutes. The pH of the reaction mixture was maintained between 6.50 and 6.90 by adding 4M KOH solution drop-wise as needed. The reaction was monitored by HPLC and was complete after 5 hours with 99.97% conversion by HPLC. The reaction mixture was stirred at 20-25° C. overnight and warmed to 30° C. for the work-up.

Step 3. (S)-1-(4,4,6,6,6-pentadeutero-5-hydroxyhexyl)-3,7-dimethyl-1H-purine-2,6(3H,7H)-dione Form 1. Intermediate 12 (1 mole equiv) was combined with water (10 volumes) and potassium carbonate (0.25 equiv) and heated to 80-85° C. for 16 hrs. The mixture was cooled to 20-25° C. and the pH adjusted to 7 with 6M aq hydrochloric acid, followed by the addition of sodium chloride. The solution was extracted with ethyl acetate and the combined organic layers were concentrated at 50-60° C., followed by the addition of n-heptane at 60° C. The slurry was cooled to 20° C., aged for 1 hr and filtered. The cake was washed with n-heptane and dried in vacuo at 45° C. to constant weight. Yield of (S)-1-(4,4,6,6,6-pentadeutero-5-hydroxyhexyl)-3,7-dimethyl-1H-purine-2,6(3H,7H)-dione Form 1 was approximately 90%.

The final product was a white solid. Deuterium incorporation as determined by ¹H-NMR at C6′ was ≧98.0%. Deuterium incorporation at C8 was <5.0%.

Form 1 was stable and did not undergo a form change when: a) exposed to 97% relative humidity for up to 12 days; exposed to 0.35 GPa pressure; or c) ground at ambient temperature (30Hz for 2hrs) or at −196° C. (25Hz for 0.5hrs).

As an alternative procedure, Form 1 may be prepared as follows: In a 3-L 3-necked RB flask, 12 (100 g) was charged followed by water (1.0 L) and K₂CO₃ (0.25 equiv). The reaction mixture was heated to 80±5° C. and monitored by ¹H NMR. The reaction was complete after 24 hours and worked up after 65 hours. The resulting product was extracted with three times with EtOAc and the solid products from the three extractions combined and re-dissolved in 5 volumes of EtOAc at 60-65° C. n-heptane (5.5 vol.) was added at 60-65° C. over 15 minutes and cooled to 20° C. over night (16 hrs). The slurry was filtered and the wet cake was washed with n-heptane (2×1 vol. to afford product Form 1 after drying at 40-50° C. A total of 92.4 g of Compound 121(S) was isolated. HPLC purity was 99.92% (AUC) and chiral selectivity was 100% to “S” enantiomer. The ¹H NMR analysis showed 99.2% of “H” at the 8-position in the 3,4,5,7-tetrahydro-1H-purine-2,6-dione ring and 99.4% of “D” at the methyl position.

As an alternative to the procedure above, intermediate 12 may be prepared according to the following two steps. In the first step, intermediate 11 is reduced with a metal hydride such as NaBH₄ and a deuterated solvent such as C₂H₅OD to form a racemic mixture of intermediate 12 and its enantiomer. In the second step, separation of 12 from its enantiomer is achieved by chromatography on chiral stationary phase. For example, a preparative Daicel Chiralpak AD column (20×250 mm) may be used for this purpose. The mobile phase may be an organic solvent or a mixture of organic solvents. Exemplary solvent mixtures comprise hexane and i-PrOH, for example, 80% hexane and 20% iPrOH with 0.1% diethylamine, or 75% hexane and 25% iPrOH along 0.1% diethylamine.

In addition to being produced by the synthesis methods described above, the Form 1 crystal can also by a) dissolving (S)-1-(4,4,6,6,6-pentadeutero-5-hydroxyhexyl)-3,7-dimethyl-1H-purine-2,6(3H,7H)-dione in neat water and lyophilizing; b) heating (S)-1-(4,4,6,6,6-pentadeutero-5-hydroxyhexyl)-3,7-dimethyl-1H-purine-2,6(3H,7H)-dione to 120° C. to cause it to melt and then cooling the molten material; c) heating (S)-1-(4,4,6,6,6-pentadeutero-5-hydroxyhexyl)-3,7-dimethyl-1H-purine-2,6(3H,7H)-dione to 220° C. to cause it to vaporize and then condensing and cooling the vaporized material. Blocks of Form 1 that were ˜100 μm in size were produced upon slow evaporation of solvents from solutions of the synthesized product dissolved in ethanol, ethyl acetate, or acetone.

### EXAMPLE 2

### Synthesis of (S)-1-(4,4,6,6,6-pentadeutero-5-hydroxyhexyl)-3,7-dimethyl-1H-purine-2,6(3H,7H)-dione Form 2

(S)-1-(4,4,6,6,6-pentadeutero-5-hydroxyhexyl)-3,7-dimethyl-1H-purine-2,6(3H,7H)-dione Form 1 (10.0 mg) was manually weighed into a 2-mL vial and combined with water (400 μL). The solution was filtered and subjected to a rapid evaporation under reduced pressure (Genevac) for 6 hrs. The obtained solid was immediately analyzed by PXRD and DSC.

The normalized PXRD analysis of Form 2 is shown in FIG. 5. That analysis shows 2-theta peaks at 4.5, 7.1, 9.1, 10.7, 11.8, 13.7, 14.1, 14.8, 18.4, 19.2, 23.0, and 24.2 degrees at ambient temperature. The DSC thermogram of Form 2 is shown in FIG. 6. The DSC thermogram shows two thermal events. The first event is an endotherm at 84° C. (onset value), immediately followed by a small exotherm, which corresponds to the conversion to Form 1. The second endotherm at 111° C. (onset) corresponds to the melting of Form 1.

### EXAMPLE 3

### Synthesis of (S)-1-(4,4,6,6,6-pentadeutero-5-hydroxyhexyl)-3,7-dimethyl-1H-purine-2,6(3H,7H)-dione Form 3

(S)-1-(4,4,6,6,6-pentadeutero-5-hydroxyhexyl)-3,7-dimethyl-1H-purine-2,6(3H,7H)-dione Form 1 (10.0 mg) was manually weighed into a 2-mL vial and combined with water (1.27 mL). The solution was filtered and subjected to a rapid evaporation under reduced pressure (Genevac) for 6 hrs. The obtained solid was immediately analyzed by PXRD and DSC.

The normalized PXRD analysis of Form 3 shown in FIG. 7 indicates 2-theta peaks at 4.9, 7.7, 8.3, 10.1, 12.1, 13.2, 14.7, 15.8, 19.5, 21.6, 23.5, 24.2, 27.7 degrees at ambient temperature.

The DSC thermogram of Form 3 is shown in FIG. 8. The DSC thermogram shows two thermal events. The first event is an endotherm at 95° C. (onset), immediately followed by a small exotherm, which corresponds to the conversion to Form 1. The second endotherm at 111° C. (onset) corresponds to the melting of Form 1.

### EXAMPLE 4

### Synthesis and Characterization of (S)-1-(4,4,6,6,6-pentadeutero-5-hydroxyhexyl)-3,7-dimethyl-1H-purine-2,6(3H,7H)-dione Form 4

(S)-1-(4,4,6,6,6-pentadeutero-5-hydroxyhexyl)-3,7-dimethyl-1H-purine-2,6(3H,7H)-dione Form 1 (20.0 mg) was manually weighed into a 2-mL vial and combined with dimethyl carbonate (500 μL) and trifluoroethanol (100 μL). The vial was vortexed until solids dissolved. The resulting solution was filtered and frozen using dried ice. The frozen vial was lyophilized for 16 hrs. The resulting material was subjected to PXRD and DSC analyses

The normalized PXRD analysis of Form 4 shown in FIG. 9 indicates 2-theta peaks at 7.5, 15.1 and 17.7 degrees.

The DSC thermogram of Form 4 is shown in FIG. 10. The DSC analysis shows two events: an endotherm at 61° C. followed immediately by a small exotherm, and an endotherm 110° C. (onset). The second endotherm corresponds to the melting of Form 1.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

We claim:

1. Form 1 polymorph of (S)-1-(4,4,6,6,6-pentadeutero-5-hydroxyhexyl)-3,7-dimethyl-1H-purine-2,6(3H,7H)-dione characterized by at least one of:
a. a powder X-ray diffraction pattern having two or more peaks expressed in degrees 2-theta±0.2° and selected from 9.3, 13.4, 18.8, 19.7, 21.8, 22.9, 23.8, and 29.5 degrees; or
b. an endotherm at 110.7±4° C.;

2. The polymorph of claim 1, characterized by a powder X-ray diffraction having peaks expressed in degrees 2-theta±0.2° at each of 9.3, 13.4, 18.8, 19.7, 21.8, 22.9, 23.8, and 29.5 degrees.

3. The polymorph of claim 2, characterized by a powder X-ray diffraction having peaks expressed in degrees 2-theta±0.2° at each of 9.3, 10.5, 11.9, 13.4, 15.5, 16.1, 18.7, 18.8, 19.7, 21.8, 22.9, 23.8, 24.3, 27.0, and 29.5 degrees.

4. The polymorph of claim 1 having at least 98% deuterium incorporation at each of the C4′ and C6′ positions, as determined by 1H-NMR.

5. The polymorph of claim 1 wherein the polymorph is substantially free of (S)-1-(4,4,6,6,6-pentadeutero-5-hydroxyhexyl)-3,7-dimethyl-8-deutero-1H-purine-2,6(3H,7H)-dione as determined by ¹H-NMR.

6. Form 2 polymorph of (S)-1-(4,4,6,6,6-pentadeutero-5-hydroxyhexyl)-3,7-dimethyl-1H-purine-2,6(3H,7H)-dione characterized by at least one of:
a. a powder X-ray diffraction pattern having two or more peaks expressed in degrees 2-theta±0.2° and selected from 4.5, 9.1, 10.7, 13.7, 14.3, 14.8, 18.4, 19.2, and 23.0 degrees; or
b. A first endotherm at 83.7±4° C.

7. The polymorph of claim 6, characterized by a powder X-ray diffraction having peaks expressed in degrees 2-theta±0.2° at each of 4.5, 9.1, 10.7, 13.7, 14.3, 14.8, 18.4, 19.2, and 23.0 degrees.

8. The polymorph of claim 7, characterized by a powder X-ray diffraction having peaks expressed in degrees 2-theta±0.2° at each of 4.5, 7.1, 9.1, 10.7, 10.9, 11.9, 13.7, 14.3, 14.8, 17.1, 18.4, 19.2, and 23.0 degrees.

9. Form 3 polymorph of (S)-1-(4,4,6,6,6-pentadeutero-5-hydroxyhexyl)-3,7-dimethyl-1H-purine-2,6(3H,7H)-dione, characterized by at least one of:
a. a powder X-ray diffraction pattern having two or more peaks expressed in degrees 2-theta±0.2° and selected from 4.9, 7.7, 8.3, 13.2, 14.7, 15.8, 19.5, 21.6, 23.5, 27.7 degrees; or
b. A first endotherm at 94.8±4° C.

10. The polymorph of claim 9, characterized by a powder X-ray diffraction having peaks expressed in degrees 2-theta±0.2° at each of 4.9, 7.7, 8.3, 13.2, 14.7, 15.8, 19.5, 21.6, 23.5, 27.7 degrees.

11. The polymorph of claim 10, characterized by a powder X-ray diffraction having peaks expressed in degrees 2-theta±0.2° at each of 4.9, 7.7, 8.3, 10.1, 12.1, 13.2, 14.7, 15.8, 19.5, 21.6, 23.5, 24.2, 27.7 degrees.

12. Form 4 polymorph of (S)-1-(4,4,6,6,6-pentadeutero-5-hydroxyhexyl)-3,7-dimethyl-1H-purine-2,6(3H,7H)-dione, characterized by at least one of:
a. An powder X-ray diffraction pattern having peaks expressed in degrees 2-theta±0.2° at each of 7.5, 15.1 and 17.7 degrees; or
b. A first endotherm at 60.7±4° C.

13. The polymorph of claim 12, characterized by a powder X-ray diffraction having peaks expressed in degrees 2-theta±0.2° at each of 7.5, 15.1 and 17.7 degrees.

14. A pharmaceutical composition comprising an effective amount of Form 1 polymorph of (S)-1-(4,4,6,6,6-pentadeutero-5-hydroxyhexyl)-3,7-dimethyl-1H-purine-2,6(3H,7H)-dione; and a pharmaceutically acceptable carrier.

15. The composition of claim 14, wherein the ratio of the amount of Form 1 to the sum of the amounts of Form 2, Form 3 and Form 4 is equal to or greater than 80:20.

16. The composition of claim 15, wherein the ratio of the amount of Form 1 to the sum of the amounts of Form 2, Form 3 and Form 4 is equal to or greater than 90:10.

17. A method of treating diabetic nephropathy in a patient comprising the step of administering to the patient a polymorph of claim 1.

18. A process for the preparation of the polymorph of claim 1, comprising (i) forming a slurry of (S)-1-(4,4,6,6,6-pentadeutero-5-hydroxyhexyl)-3,7-dimethyl-1H-purine-2,6(3H,7H)-dione in ethyl acetate and n-heptane, and (ii) cooling the slurry to a temperature sufficiently low to form the polymorph.

19. A process for the preparation of the polymorph of claim 1, comprising dissolving (S)-1-(4,4,6,6,6-pentadeutero-5-hydroxyhexyl)-3,7-dimethyl-1H-purine-2,6(3H,7H)-dione in a solvent selected from ethanol, ethyl acetate, and acetone and evaporating the solvent.

20. The process of claim 19, further comprising evaporating the solvent over 2-28 days at ambient temperature to form the polymorph.

21. The polymorph of claim 1, wherein the polymorph is substantially free of amorphous (S)-1-(4,4,6,6,6-pentadeutero-5-hydroxyhexyl)-3,7-dimethyl-1H-purine-2,6(3H,7H)-dione.
